(19) United States
(12) Patent Application Publication
(10) Pub. No.: US 2008/0071081 A1
(43) Pub. Date: Mar. 20, 2008

(54) Method for Catalytic Hydrogenation Purification of Lactam Containing Chlorolactam Impurities

(76) Inventors: Eric Lacroix, Amberieuxx D'Azergues (FR); Serge Hub, Villeurbanne (FR); Philippe Bonnet, Lyon (FR); Michel Devic, Lyon (FR)

Correspondence Address: ARKEMA INC.;PATENT DEPARTMENT - 26TH FLOOR, 2000 MARKET STREET, PHILADELPHIA, PA, 19103-3222, US

(21) Appl. No.: 11/632,339

(22) PCT Filed: Jul. 12, 2005
(86) PCT No.: PCT/FR05/01794

§ 371 Date: Jan. 12, 2007

(30) Foreign Application Priority Data
Jul. 16, 2004 (FR) 04 07 918

(51) Int. Cl. C07D 225/00

(52) U.S. Cl. 540/451

(57) ABSTRACT

The invention concerns a method for purifying cyclic lactams containing 6 to 12 carbon atoms and containing chlorolactams as impurities, by hydrogenation reaction in the presence of a metal catalyst, a solvent and a compound having a radical —NH₂, —NH— or —N< radical is that of a HCl capturing amino generated during the hydrgantation reaction, and the organic solvent is a solvent capable of solublizing the aminated hydrochloride formed by the capture of HCl by the amino; and the mol ratio represented by the percentage of amine expressed in mol over the percentage of elementary organic chlorine in the form of chlorolactam expressed in mols, is at least 0.5.

### FIELD OF THE INVENTION

The present invention relates to a method for purifying cyclic lactams containing chlorolactam impurities by catalytic hydrogenation reaction.

PRIOR ART AND THE TECHNICAL PROBLEM

The preparation of high quality polyamides requires the use of very pure lactams. Impurities in low quantity, barely detectable by analysis, cause a reduction in the quality of the polyamides.

Polyamides prepared from impure lactams thus show a tendency towards yellowing and have a broad spectrum of molecular weights.

In addition, the nature of the impurities depends on the method of synthesizing the lactam used. Thus, the lactam prepared by photooximation contains chlorinated impurities characteristic of this type of method (chlorolactams), whose presence degrades the properties of the polymers manufactured.

The elimination of these chlorinated impurities is therefore essential. One of the purification techniques well known to a person skilled in the art is treating the lactam with hydrogen over hydrogenation catalysts. This reaction eliminates chlorine in the form of HCl and thus makes it possible to convert the chlorolactams to lactams.

However, according to Patent JP Sho 47-37632/72, conventional hydrogenation catalysts such as Raney nickel are deactivated quite quickly. According to the authors, the cause of this deactivation is directly linked to the low solubility of the HCl coproduct in conventionally used reaction media (lactam/organic solvents), blocking the active sites of the catalyst.

The use of a manganese-modified nickel catalyst, as well as the addition of an alkaline hydroxide (to neutralize the HCl produced) makes it possible to improve the lifetime of the catalyst system, without however being totally satisfactory.

The Patent JP Sho 47-37633/72 proposes a catalyst based on Ni and on BaF₂ deposited onto SiO₂. The lifetime of this catalyst is better than that of the catalyst cited as reference, but it has nevertheless a measurable deactivation.

The hydrogenation purification methods require solvents that are inert with respect to hydrogen, which prevents amongst other things the use of aromatic compounds, which are in general much better solvents for lactam. The Patent JP Sho 46-23743/71 describes however a hydrogenation method with aromatic solvents. In order to avoid hydrogenating the solvent, it is recommended to add an amine that is soluble in the aromatic solvent medium. The amine is only used to inhibit the hydrogenation of the solvent and may therefore be used in small quantities relative to the chlorinated impurities present. Furthermore, an alkaline hydroxide is added to the medium in an at least stoichiometric quantity relative to the initial organic chlorine in order to neutralize the acidity produced by the reaction.

It appears therefore that the lactam purification by hydrogenation requires the addition of an alkaline hydroxide solution and optionally of an amine when the solvent for the lactam is an aromatic solvent. However, the presence of these additives does not prevent the deactivation of the catalysts used.

Without wanting to be tied to one theory, it is conceivable that the deactivation of the catalysts could probably be carried out by crystallization of the chlorinated salts formed on the catalyst.

There is therefore a need to improve these methods for purifying lactams by hydrogenation of the chlorolactams and in particular to prevent the deactivation of the hydrogenation catalysts used.

### SUMMARY OF THE INVENTION

An objective of the invention is to propose a new method for purifying cyclic lactams containing 6 to 12 carbon atoms and containing chlorolactams as impurities, by hydrogenation reaction in the presence of a metal catalyst, a solvent and a compound having a —NH₂, —NH— or —N< residue in which:
the —NH₂, —NH— or —N< residue is that of an amine that has captured the HCl generated during the hydrogenation reaction; and
the organic solvent is a solvent capable of dissolving the amine hydrochloride formed by the capture of HCl by the amine; and
the molar ratio represented by the % of amine expressed in mols over the % of elementary organic chlorine in the form of chlorolactam expressed in mols, is greater than or equal to 0.5.

According to one embodiment, the molar ratio is between 1 and 100 and preferably between 5 and 40.

According to one embodiment, the amine is chosen from the group of linear or branched primary, secondary and tertiary alkylamines R₁NH₂, R₁R₂NH and R₁R₂R₃N with R₁═CₙH₂ₙ₊₁ n=2 to 12, R₂═CₘH₂ₘ₊₁ m=1 to 3 and R₃═CₚH₂ₚ₊₁ p=1 to 3, preferably the amine is tert-octylamine.

According to one embodiment, the organic solvent is chosen from the group made up by acyclic or cyclic saturated aliphatic hydrocarbon-based solvents, preferably cyclohexane, aromatic solvents and ionic liquids.

The invention also relates to a method for purifying cyclic lactams containing 6 to 12 carbon atoms and containing chlorolactams as impurities, by hydrogenation reaction in the presence of a metal catalyst, a solvent and a compound having a —NH— residue in which, the —NH— residue is that of the lactam present or formed during the hydrogenation reaction and the organic solvent is an ionic liquid capable of dissolving the lactam hydrochloride formed.

According to a preferred embodiment an organic solvent is used in the purification method, which is an ionic liquid chosen from the group made up by ionic liquids comprising a dialkylimidazolium derivative of formula I or II 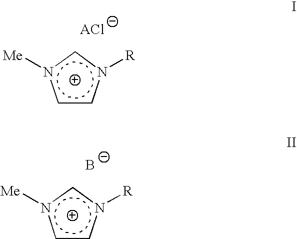
in which
R is a CH₃, —(CH₂)ₙ—CH₃, —CH₂—CₙF₂ₙ₊₁ or phenyl group with n being an integer between 1 and 10, and
A represents AlCl₃, SbCl₃F₂, TaCl₅, TaF₅, NbCl₅, TiCl₄,
B represents SbF₆, BF₄, PF₆, CF₃SO₃.

According to one embodiment, the ionic liquid is formed by the association between the dialkylimidazolium cation of formula I with R as defined above and a hexafluoroborate BF₆ anion or else a hexafluorophosphate PF₆ anion.

According to one embodiment, the method is operated at a temperature between 100 and 250° C.

According to one embodiment, the lactam to be purified is lauryl lactam or caprolactam.

According to one embodiment, the lactam to be purified contains up to 5% by weight of chlorolactam.

According to one embodiment, the hydrogenation step is carried out by continuously passing a flow of lactam, in a solvent optionally containing the amine, with hydrogen over a catalyst.

### DETAILED DESCRIPTION OF THE INVENTION

The method for purifying cyclic lactams containing 6 to 12 carbon atoms is carried out by eliminating the corresponding chlorolactams by hydrogenation reaction in the presence of a metal catalyst, an amine to capture the HCl formed during the hydrogenation reaction in the form of the amine hydrochloride and an organic solvent capable of dissolving the amine hydrochloride formed.

This method improves the yields and the conversion rate of the chlorolactam to lactam.

The lactam to be purified may contain up to 5% by weight of chlorolactam impurities relative to the weight of the lactam used, preferably up to 1%, and more particularly up to 0.1% by weight.

The amine chosen is such that its salt with the hydrochloric acid is totally or partially soluble in the reaction solvent used. The solubility of the salt formed is especially dependent on the quantity of chlorolactam to be eliminated. Thus, in order to carry out the purification it will be possible to choose, in a suitable manner, the concentration of the lactam to be purified in the chosen solvent, so as not to exceed the solubility of the amine salt formed.

The presence of such an amine makes it possible to avoid the deactivation of the catalyst.

Indeed the amine hydrochloride formed is not deposited on the catalyst on account of its solubility in the reaction solvent used and therefore the catalyst is not, or only slightly, deactivated.

The quantity of amine used depends on the quantity of elementary organic chlorine present in the medium in the form of chlorolactam.

Thus a molar ratio is defined corresponding to the ratio between the % of amine expressed in mols and the % of chlorolactam expressed in mols of elementary organic chlorine initially present in the solution.

The molar ratio as defined above is greater than or equal to 0.5, preferably between 1 and 100, even between 5 and 40.

Any amine is used in the method, whose hydrochloride is soluble or partially soluble in an organic medium, such as the linear or branched primary, secondary and tertiary alkylamines R₁NH₂, R₁R₂NH and R₁R₂R₃N with R₁═CₙH₂ₙ₊₁ n=2 to 12, R₂═CₘH₂ₘ₊₁ m=1 to 3 and R₃═CₚH₂ₚ₊₁ p=1 to 3.

According to one particular embodiment, the amine is also totally or partially soluble in the organic solvent used.

Primary amines are preferred and in particular tert-octylamine [(CH₂)₃C—CH₂—C(CH₃)₂NH₂].

The organic solvent to dissolve the amine hydrochloride is in general chosen from the group made up by acyclic or cyclic saturated aliphatic hydrocarbon-based solvents, aromatic solvents and ionic liquids.

The acyclic or cyclic saturated aliphatic hydrocarbon-based solvents are chosen from linear or branched hexane, heptane, octane, nonane, decane, undecane or dodecane, optionally substituted cyclohexane, cycloheptane, cyclooctane, cyclodecane or cyclododecane and mixtures thereof, and preferably the organic solvent chosen is cyclohexane.

The aromatic solvents are chosen from benzene, toluene, ethylbenzene, xylenes, cumene and mixtures thereof.

The ionic liquids used in the invention are described for example in Patent Application No. WO 01/83353 (page 1 to page 4 and the examples).

Ionic liquids comprising a dialkylimidazolium derivative of formula I or II are preferred 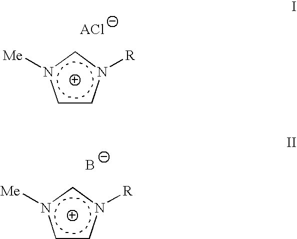
in which
R is a CH₃, —(CH₂)ₙ—CH₃, —CH₂—CₙF₂ₙ₊₁ or phenyl group with n being an integer between 1 and 10, and
A represents AlCl₃, SbCl₃F₂, TaCl₅, TaF₅, NbCl₅, TiCl₄,
B represents SbF₆, BF₄, PF₆, CF₃SO₃.

The ionic liquids formed by the association between the dialkylimidazolium cation of formula I with R as defined above and a hexafluoroborate BF₆ anion or else a hexafluorophosphate PF₆ anion are more preferred.

According to a preferred embodiment, solvents of the group made up by acyclic or cyclic saturated aliphatic hydrocarbon-based solvents, or else the ionic liquids cited above, are used.

Any hydrogenation catalyst based on metals deposited on a support or in bulk may be used in the method, but it is preferred to use a catalyst based on Ni or on Pd deposited on alumina, silica, silica-alumina or carbon.

The operating temperature is in general below the decomposition temperature of the amine salt, and preferably between 100 and 250° C.

The pressure may be between 1 and 100 bar, preferably between 5 and 50 bar, and more particularly between 10 and 40 bar.

The purification method according to the invention may be carried out in order to purify cyclic lactams containing chlorolactams as impurities, in particular C₆ (caprolactam) or C₁₂ (lauryl lactam) lactams, base monomers in the synthesis of polyamides.

Indeed, the use of such a method for purifying lactams makes it possible to eliminate the chlorolactams by converting them to lactams and, due to the presence of amine, to prevent the capture of the HCl generated during the hydrogenation by the lactam itself. Consequently, this method makes it possible to avoid depositing the lactam hydrochloride on the catalyst, in the very frequent cases when this salt is insoluble in the reaction medium. Thus the presence of the amine makes it possible to avoid the deactivation of the catalyst and to improve the conversion yields of the chlorolactams to lactams.

According to another embodiment, the invention proposes a method for purifying cyclic lactams containing 6 to 12 carbon atoms having chlorolactams as impurities, by hydrogenation reaction in the presence of a metal catalyst, a solvent and a compound having a —NH— residue in which, the —NH— residue is that of the lactam present or formed during the hydrogenation reaction and the organic solvent is chosen from the ionic liquids as defined previously. The ionic liquid is capable of dissolving the lactam hydrochloride formed.

The presence of the ionic liquid makes it possible to avoid deactivation of the catalyst and to improve the conversion yields of the chlorolactams to lactams.

According to a preferred embodiment, the metal catalyst used here is Pd/C or Ni/SiO₂.

The preferred ionic liquid is bmimPF₆ in which the lactam hydrochloride, which is formed during the hydrogenation reaction between the HCl and the lactam, is soluble at a proportion of 3% at 25° C. and 35% at 100° C. Therefore, the dissolved lactam hydrochloride is not deposited on the catalyst. The lifetime of the catalyst and the conversion yields of the chlorolactam to lactam are thus improved.

According to another embodiment, the method of purifying the lactams is a continuous method in which a flow of the lactam to be continuously purified, in the solvent optionally containing the amine, is passed with hydrogen over a catalyst.

The hourly space velocity may be between 0.1 and 10 h⁻¹, but preferentially is operated between 1 and 5 h⁻¹.

### EXAMPLES

The following examples illustrate the present invention without however limiting the scope thereof. The examples called comparative examples correspond to tests without amine.

### Comparative Example 1c

A solution of cyclohexane with 10% by weight of lauryl lactam (PM 197) containing 0.085% by weight of chlorine in the form of chlorolauryl lactam (being 0.024 mol % of elementary organic chlorine) was prepared. This solution was then fed continuously (86 g/h) into the reactor with hydrogen (0.5 l/h) under a pressure of 20 bar and a temperature T of 150° C. in a reactor containing a quantity Qc of 40 ml of commercial hydrogenation catalyst Ni—NiO—SiO₂—Al₂O₃.

The mixture resulting from the reactor was first decanted to separate the liquid and gas phases. The lauryl lactam was then recovered after evaporation of the solvent. The crystals obtained were washed with water to eliminate the salts produced.

The organic chlorine initially attached to a carbon atom of the lauryl lactam was discovered after the hydrogen treatment in the form of HCl linked to the lactam itself. Washing with water allowed this HCl to be recovered which could then be measured in the form of chlorides.

The quantity of elementary organic chlorine initially present in the lactam in the form of chlorolauryl lactam, the quantity of residual elementary organic chlorine remaining attached to the lactam and the quantity of chlorides recovered in the water washings were measured, everything expressed as % by weight of elementary chlorine.

The conversion of the chlorolactams was determined by the ratio:

$$\frac{\% initial organic chlorine - \% residual organic chlorine}{\% initial organic chlorine}$$

The chlorine balance was determined by the ratio:

$$\frac{\% residual organic chlorine + \% recovered chlorides}{\% initial chlorine}$$

The possible discrepancy in this chlorine balance (difference with respect to 100%) is synonymous with the accumulation of product on the catalyst and therefore with the decrease in the lifetime of the catalyst.

The molar ratio corresponding to the ratio between the % of amine expressed in mols of tert-octylamine (PM=129) and the % of chlorolauryl lactam expressed in mols of elementary organic chlorine initially present in the solution was determined.

The following table summarizes the results of the conversion of chlorolactam to lactam and also the chlorine balance as a function of the various parameters T, molar ratio, % of amine by weight, quantity and type of catalyst and continuous operating time of the reactor.

| | | Amine/elementary | % amine | | | | Cl |
|---|---|---|---|---|---|---|---|
| | T | chlorine molar | by | Type/quantity | % | | balance |
| Tests | ° C. | ratio | weight | of catalyst | conversion | | % |
| Comparative | 150 | 0 | 0 | Ni—NiO—SiO₂—Al₂O₃/40 ml | 79 | | 22 |
| example 1c | | | | | | | |
| Example 1 | 150 | 6.2 | 0.2 | Ni—NiO—SiO₂—Al₂O₃/40 ml | 71 | | 40 |
| Example 2 | 150 | 35.2 | 1 | Ni—NiO—SiO₂—Al₂O₃/40 ml | 53 | | 80 |
| Comparative | 180 | 0 | 0 | Pd/C-0.5% | 40 | | 71 |
| example 3c | | | | | | | |
| Example 3 | 180 | 38.5 | 1 | Pd/C-0.5% | 85 | | 100 |
| Comparative | 180 | 0 | 0 | Ni—NiO—SiO₂—Al₂O₃/80 ml | 21 h | 97 | 4 |
| example 4c | | 0 | | | 54 h | 97 | 13 |
| | | 0 | | | 70 h | 92 | 19 |
| | | 0 | | | 75 h | 92 | 16 |
| | | 0 | | | 108 h | 89 | 16 |
| | | 0 | | | 160 h | 81 | 26 |
| Example 4 | 180 | 33.2 | 1.2 | Ni—NiO—SiO₂—Al₂O₃/80 ml | 20 h | 97 | 98 |
| | | 33.2 | | | 250 h | 96 | 97 |

### Example 1

In the reaction conditions described in example 1c, a solution of cyclohexane containing 0.2% by weight of tert-octylamine (0.155 mol %) and 10% by weight of lauryl lactam was supplied. The initial quantity of organic chlorine in the lactam was 0.09% by weight (being 0.025 mol % of elementary chlorine in the solution) and the residual content after purification was 0.026% by weight. The quantity of chlorides measured in the water washings was 0.01% by weight. The conversion of the chlorolactams was then 71%.

The chlorine balance was 40%.

### Example 2

In the reaction conditions described above, a solution of cyclohexane containing 1% by weight of tert-octylamine (0.775 mol %) and 10% by weight of lauryl lactam was supplied. The initial quantity of organic chlorine in the lactam was 0.077% by weight (being 0.022% in mols in the solution) and the residual content after purification was 0.036% by weight. The quantity of chlorides measured in the water washings was 0.025% by weight.

The conversion of the chlorolactams was then 53% and the chlorine balance was 80%.

### Comparative Example 3c

A solution of cyclohexane with 10% of lauryl lactam containing 0.0565% by weight of chlorine was prepared. This solution was then fed continuously (82 g/h) into the reactor with hydrogen (0.5 l/h) under 20 bar and 180° C. in a reactor containing 40 ml of commercial hydrogenation catalyst 0.5% Pd/C.

The mixture resulting from the reactor was first decanted in order to separate the liquid and gas phases. The lauryl lactam was then recovered after evaporation of the solvent. The crystals obtained were washed with water to eliminate the salts produced. The quantity of residual organic chlorine in the lactam (obtained by gas chromatography GC) was then 0.0337% by weight. The quantity of chlorides measured in the water washings was 0.01% by weight.

### Example 3

In the reaction conditions described above, a solution of cyclohexane containing 1% by weight of tert-octylamine (0.775 mol %) and 10% of lauryl lactam was supplied. The initial quantity of organic chlorine in the lactam was 0.073% by weight (being 0.020 mol % in the solution) and the residual content after purification was 0.011% by weight.

The quantity of chloride measured in the water washings was 0.063% by weight.

The conversion of the chlorolactams was then 85% and the chlorine balance was 100%.

### Comparative Example 4c

A solution of cyclohexane with 10% by weight of lauryl lactam containing 0.103% by weight of chlorine was prepared. This solution was then fed continuously (82 g/h) into the reactor with hydrogen (0.5 l/h) under 20 bar and 180° C. in a reactor containing 80 ml of commercial hydrogenation catalyst Ni—NiO—SiO₂—Al₂O₃.

The mixture resulting from the reactor was removed at various reaction times. The mixture was first decanted in order to separate the liquid and gas phases. The lauryl lactam was then recovered after evaporation of the solvent. The crystals obtained were washed with water to eliminate the salts produced.

The performance of the reaction is evaluated here as a function of the contact time of the reactants.

| | Residual | | | |
|---|---|---|---|---|
| Time | organic Cl | Chlorides | % conversion | Cl balance % |
| 21 hrs | 0.003% | 0.001% | 97% | 4 |
| 54 hrs | 0.003% | 0.010% | 97% | 13 |
| 70 hrs | 0.008% | 0.012% | 92% | 19 |
| 75 hrs | 0.008% | 0.009% | 92% | 16 |
| 108 hrs | 0.011% | 0.006% | 89% | 16 |
| 163 hrs | 0.020% | 0.007% | 81% | 26 |

The quantity of chlorides recovered at the outlet of the reactor only makes up one part (<10%) of the chlorine initially present in the lactam in the form of organic chlorine. The chlorides formed accumulate on the catalyst leading to a deterioration in its performance.

### Example 4

In the reaction conditions described above, a solution of cyclohexane containing 1.2% by weight of tert-octylamine (0.930 mol %) and 10% of lauryl lactam was supplied. The initial quantity of organic chlorine in the lactam was 0.1% by weight (being 0.028 mol % in the solution). After running for 20 hours, the content of residual organic chlorine after purification was 0.003% by weight. The quantity of chloride measured in the water washings was 0.095% by weight. The conversion of the chlorolactams was then 97% and the chlorine balance was 98%. After running for 250 hours, the conversion was 96% with a chlorine balance of 97%.

### Example 5

### With Ionic Liquid and without Amine

A stainless steel autoclave, under an argon atmosphere in order to be free of moisture, was charged with:
50 g of bmimPF6, 2.64 g of Pd/C catalyst and 5 g of chlorinated lactam (0.17% of total chlorine).

The autoclave was pressurized with 20 bar of hydrogen and the reaction medium was heated at 100° C. for 6 hours with stirring and under 20 bar of hydrogen.

After the reaction, 50 g of methanol was added in order to fluidize the medium and it was filtered to isolate the catalyst.

The methanol was evaporated under vacuum and the crystallized lactam was filtered and washed with a small amount of acetonitrile and with water, then dried in an oven at 80° C.

The total chlorine content was 0.005% instead of 0.17% before treatment, being a conversion rate of the chlorolactam to lactam of 97%.

### Comparative Example 5c

The procedure of example 5 was followed exactly but replacing the ionic liquid with 50 g of cyclohexane.

The total chlorine content of the lactam after treatment and washing with water was 0.13% instead of 0.17% before treatment, i.e. a degree of conversion of chlorolactam to lactam of 23.5%.

1-13. (canceled)

1. A method for purifying cyclic lactams containing 6 to 12 carbon atoms and containing chlorolactams as impurities, comprising carrying out a hydrogenation reaction in the presence of a metal catalyst, a solvent and a compound having a —NH— residue in which wherein, the —NH— residue is that of the lactam present or formed during the hydrogenation reaction and the organic solvent is an ionic liquid capable of dissolving the lactam hydrochloride formed.

2. The method as claimed in claim 1, wherein the organic solvent is an ionic liquid chosen from the group made up by ionic liquids comprising a dialkylimidazolium derivative of formula I or II 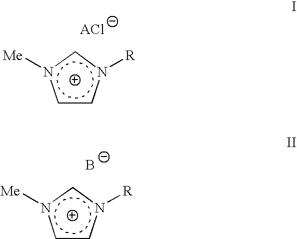
in which
R is a CH₃, —(CH₂)ₙ—CH₃, —CH₂—CₙF₂ₙ₊₁ or phenyl group with n being an integer between I and 10, and
A represents AlCl₃, SbCl₃F₂, TaCl₅, TaF₅, NbCl₅, TiCl₄,
B represents SbF₆, BF₄, PF₆, CF₃SO₃.

3. The method as claimed in claim 2, wherein the ionic liquid is formed by the association between the dialkylimidazolium cation of formula I with R and a hexafluoroborate BF₆ anion or else a hexafluorophosphate PF₆ anion.

4. The method as claimed in claim 1, wherein the temperature is between 100 and 250° C.

5. The method as claimed in claim 1, wherein the lactam is lauryl lactam or caprolactam.

6. The method as claimed in claim 1, wherein the lactam to be purified contains up to 5% by weight of chlorolactam.

7. The purification method as claimed in claim 1, wherein the hydrogenation step is carried out by continuously passing a flow of lactam, in a solvent optionally containing the amine, with hydrogen over a catalyst.
